(19) United States
(12) Patent Application Publication
(10) Pub. No.: US 2016/0008225 A1
(43) Pub. Date: Jan. 14, 2016

(54) Medication Applicator

(71) Applicant: Sammi Molvi, Corvallis, OR (US)

(72) Inventor: Sammi Molvi, Corvallis, OR (US)

(21) Appl. No.: 14/329,195

(22) Filed: Jul. 11, 2014

(51) Int. Cl. A61J 7/00; A61M 35/00

(52) U.S. Cl. CPC A61J 7/0053; A61M 35/003

(57) ABSTRACT

A medication applicator for accurately and precisely dispensing medication is provided herein. The applicator can dispense any non-solid medication that does not require subcutaneous administration. Most topical and oral medications can be dispensed from the medication applicator. The medication applicator has a twist grip causes a predetermined dosage amount to be dispensed. When the twist grip is rotated, an internal plunger advances within the applicator causing medication to be dispensed from the applicator's tip. Some embodiments of the invention are one-use, disposable products. When not in use, a threaded cap screws onto the applicator's tip keeping it protected from accidental damage.

### CROSS-REFERENCES TO RELATED APPLICATIONS

This application claims priority to U.S. Provisional Patent Application Ser. No. 61/846,023 filed on Jul. 14, 2013 entitled “Medication Applicator”, the disclosure of which is hereby incorporated by reference.

### FIELD OF THE INVENTION

The present invention relates to the dispensation of medication, and more specifically, to a syringe-type device that dispenses exact quantities of medication.

### BACKGROUND OF THE RELATED ART

Medication applicators are well known within the art. Numerous devices have been used throughout history to administer medication to a patient. One of the most common types of medication applicator is a medical syringe. Medical syringes have been adapted to provide for the delivery of medications, usually in liquid form, as an injection. Refinements in the accuracy and consistency of medical syringes have allowed them to deliver doses in ever-exacting amounts.

A typical medical syringe is a simple pump comprising a plunger that tightly fits within a transparent tube known as a barrel. An elastomeric or rubber platform is attached to one end of the plunger that creates a movable fluid-tight seal within the barrel. A thumb rest is often provided at the other end of the plunger so that a user can depress the plunger with their thumb. A flange or finger rest is sometimes installed along the open-end of the barrel that allows a user to exert a contracting pressure between the thumb that is on their plunger and their fingers that are on the barrel when the plunger is depressed. However, some contemporary syringes known as pen-type syringes are self-contained and do require finger manipulation by the user to dispense medication.

Most syringes are fitted with a hypodermic needle at one end of the barrel through with medication flows when the plunger is depressed. Alternatively, a nozzle, tubing, or an open-end can take the place of the hypodermic needle. These medical syringes can come pre-filled, where a set dose of medication is already installed within the barrel, or empty, where they are filled with a desired amount of medication from a reservoir such as a vial or bottle at the time of administration.

When medication is administered as an injection, the plunger is depressed and medication flows out of the barrel, through a hypodermic needle, and into a patient. Commonly, the hypodermic needle is inserted into the patient prior to depressing the plunger. When used to administer injections, medication can be delivered into a patient's blood stream, intramuscularly, or subcutaneously as is common with insulin injections. Topical or oral administration of medication is performed much in the same was as an injection where medication flows out of the barrel when the plunger is depressed, but instead flows out through a nozzle instead of a hypodermic needle.

Markings along the barrel of a syringe indicate the interior volume. When a plunger is made to align with these markings, the remaining volume within the barrel can be roughly determined. These markings also allow the amount of medication within the barrel to be estimated so that dosages can be controlled. A dose of medication is dispensed when the user causes the plunger to move from a marking that indicates a starting volume to a marking that indicates a final volume. The difference between the start and final volume is the amount of medication that was dispensed.

To administer medication, the user first determines the amount of medication to be dispensed then depresses the plunger causing it to travel along the barrel. The farther the plunger travels, the more medication is dispensed.

Significant problems exist when attempting to dispense exact doses from traditional plunger-based syringes. A user's dexterity is required to ensure that the plunger travels the proper distance. Too far and too much medication is administered, not far enough and not enough medication is administered. This is a particular problem when a user must also load an empty syringe by drawing medication into it from a medication reservoir. Similarly, it is a problem with pre-loaded syringes where only a portion of the medication must be dispensed. With pre-loaded syringes, users may not be able to administer an exact amount of medication during dispensation.

Furthermore, syringes are commonly used by patients that have to administer their own medications. Depending on the location where the medication must be administered, and the motor skills of the patient, it may be difficult for a patient to self-administer a proper dose. It is possible that a patient can injure themselves by administering too much or too little of the medication.

The prior art does present syringes that dispense specific amounts of medication. These syringes are metered, ratchet-based systems that operate by dispensing medication in pre-determined portions set by operating an adjustment knob. During dispensation, a user twists a knob, usually located at one end of the syringe, to set the amount of medication to be dispensed. After setting the dosage, the user depresses a plunger or presses a button that releases a spring-loaded plunger and the exact amount of medication is dispensed. Unfortunately, these ratchet-based syringes are used with needle-based syringes. The prior art does not present a ratchet-based medication dispensation device intended to dispense topical or oral medications. Additionally, the prior art does not present a ratchet-based medication dispensation device that is capable of dispensing non-liquid medications such as creams, ointments, or gels.

There exists a strong need within the prior art for a device that can administer exact doses of topical or oral medication. The present invention meets this need by providing a pen-type syringe that accurately and precisely delivers measured amounts of medication.

### BRIEF SUMMARY OF THE INVENTION

The present invention provides a medication applicator that provides accurate and precise doses of non-subcutaneous medication. The medication may be topical or oral medication, or any other type of medication that does not require needle-based subcutaneous administration. The applicator provides the medication when a user rotates a twist grip. The rotation of the twist grip causes a plunger to advance within the applicator. Medication is dispensed from the applicator's tip. A cap is provided to protect the tip when the applicator is not in use. The user controls how much medication is dispensed by controlling how far the grip is rotated. Typically, a minimum dose is dispensed after a half turn which, in some embodiments of the present invention, is 0.05 mL +/−5%.

By providing this improved applicator, many benefits are realized over the prior art. Dosages are dispensed in measured and controlled amounts, the medication is safely stored within the applicator, and the applicator provides a quick and convenient means for dispensing the medication. Furthermore, the applicator is eminently portable making it convenient to keep with the user at all times.

The preceding brief description is intended to merely outline some functions and advantages of the present invention. The following disclosure will set forth other functions and advantages of the present invention along with novel features that distinguish the present invention from the prior art. It is to be understood that the following disclosure is by no means intended to limit the scope of the present invention or any of its embodiments. It is also to be understood that the accompanying illustrations are presented for descriptive purposes only and similarly are not intended to limit the scope of present invention or any of its embodiments. The following disclosure and accompanying illustrations may describe various features of novelty that characterize the invention. The invention does not reside any particular feature when taken in the singular, but in the combination of features as described herein.

Other novel features which are characteristics of the invention, as to organization and method of operation, together with further and advantages thereof will be better understood from the following description considered in connection with the accompanying figures, in which preferred embodiments of the invention are illustrated by way of example. It is to be expressly understood, however, that the figures are for illustration and description only and are not intended as a definition of the limits of the invention. The various features of novelty which characterize the invention are pointed out with particularity in the following description. The invention resides not in any one of these features taken alone, but rather in the particular combination of all of its structures for the functions specified.

### BRIEF DESCRIPTION OF THE FIGURES

FIG. 1A shows a side view of a screw cap as according to one embodiment of the present invention;

FIG. 1B shows a bottom view of a screw cap as according to one embodiment of the present invention;

FIG. 1C shows a side elevated view of a screw cap as according to one embodiment of the present invention;

FIG. 2A shows a side view of an applicator tip as according to one embodiment of the present invention;

FIG. 2B shows a bottom view of an applicator tip as according to one embodiment of the present invention;

FIG. 2C shows a top view of an applicator tip as according to one embodiment of the present invention;

FIG. 3A shows a side elevated view of a medication applicator barrel as according to one embodiment of the present invention;

FIG. 3B shows a side view of a medication applicator barrel as according to one embodiment of the present invention;

FIG. 4A shows a side elevated view of a plunger as according to one embodiment of the present invention;

FIG. 4B shows a side view of a plunger as according to one embodiment of the present invention;

FIG. 5A shows a side elevated view of a ratchet disk as according to one embodiment of the present invention;

FIG. 5B shows a first side view of a ratchet disk as according to one embodiment of the present invention;

FIG. 5C shows a second side view of a ratchet disk as according to one embodiment of the present invention;

FIG. 5D shows a top view of a ratchet disk as according to one embodiment of the present invention;

FIG. 6 shows a front view of a spring as according to one embodiment of the present invention;

FIG. 7A shows an elevated side view of a threaded rod as according to one embodiment of the present invention;

FIG. 7B shows a first side view of a threaded rod as according to one embodiment of the present invention;

FIG. 7C shows a second side view of a threaded rod as according to one embodiment of the present invention;

FIG. 8A shows a side elevated view of a twist grip as according to one embodiment of the present invention;

FIG. 8B shows a side view of a twist grip as according to one embodiment of the present invention; and

FIG. 9 shows a component view of the assembly of a medication applicator as according to one embodiment of the present invention.

A further understanding of the present invention can be obtained by reference to a preferred embodiment set forth in the accompanying description. Although the illustrated embodiments are merely exemplary of methods for carrying out the present invention, both the organization and method of operation of the invention, in general, together with further objectives and advantages thereof, may be more easily understood by reference to the illustrations and the following description. The figures are not intended to limit the scope of this invention, but merely to clarify and exemplify the invention.

Certain figures contain labels, measurements, or other alphanumeric indicators. None of the aforementioned are intended to limit the scope of the invention, but are included merely to clarify and exemplify the invention.

### DETAILED DESCRIPTION OF THE INVENTION

In the following detailed description, reference is made to specific embodiments in which the invention may be practiced. These embodiments are described in sufficient detail to enable those skilled in the art to practice the invention. It is to be understood that the various embodiments of the invention, although different, are not necessarily mutually exclusive. Furthermore, a particular feature, structure, or characteristic described herein in connection with one embodiment may be implemented within other embodiments without departing from the scope of the invention. In addition, it is to be understood that the location or arrangement of individual elements within each disclosed embodiment may be modified without departing from the scope of the invention. The following detailed description is, therefore, not to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the terms “embodiment(s) of the invention”, “alternative embodiment(s)”, and “exemplary embodiment(s)” do not require that all embodiments of the method, system, and apparatus include the discussed feature, advantage or mode of operation. The following description of the preferred embodiment is merely exemplary in nature and is in no way intended to limit the invention, its application, or use.

There has thus been broadly outlined the more important features of the invention in order that the detailed description thereof that follows may be better understood, and in order that the present contribution to the art may be better appreciated. There are, of course, additional features of the invention that will be described hereinafter and which will form additional subject matter. Those skilled in the art will appreciate that the conception upon which this disclosure is based may be readily utilized as a basis for the designing of other structures, methods and systems for carrying out the purposes of the present invention. It is important, therefore, that any embodiments of the present invention be regarded as including such equivalent constructions insofar as they do not depart from the spirit and scope of the present invention.

In a manner described below, some or all of the present invention may be designed, tested, or manufactured by programs that are executed by a computer. The term “computer” as used herein includes any device that electronically executes one or more programs, such as personal computers (PCs), hand-held devices, multi-processor systems, microprocessor-based programmable consumer electronics, network PCs, minicomputers, mainframe computers, routers, gateways, hubs and the like. The term “program” as used herein includes applications, routines, objects, components, data structures and the like that perform particular tasks or implement particular abstract data types. The term “program” as used herein further may connote a single program application or module or multiple applications or program modules acting in concert. The design, testing, or manufacturing aspects of the invention also may be employed in distributed computing environments, where tasks are performed by remote processing devices that are linked through a communications network. In a distributed computing environment, programs may be located in both local and remote memory storage devices.

Further, the purpose of the Abstract herein is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The Abstract is neither intended to define the invention of this application nor is it intended to be limiting as to the scope of the invention in any way.

Referring now to the present invention, there is introduced a medication applicator that accurately and precisely dispenses doses of topical or oral medication. For the purpose of clarity, all like elements will have the same numbering and designations in each of the Figures. The terms “medication applicator”, “applicator”, “invention”, and “present invention” may be used interchangeably. In addition to the functions, features, components, and abilities of the invention already discussed in this specification, the invention may also have, but not be limited to, the following features contained within the description below.

The present invention solves the shortcomings of the prior art by providing a medication applicator that provides accurate and precise doses of non-subcutaneous medications such as topical or oral medications. The preferred embodiments described below set forth the present invention in greater detail.

Several preferred embodiments of the medication applicator are discussed in this section. However, the invention is not limited to these embodiments. A medication applicator, as according to the present invention, is any device that provides accurate and precise doses of medication that do not require injection. The medication may be topical or oral medication, or any other type of medication that does not require needle-based injection.

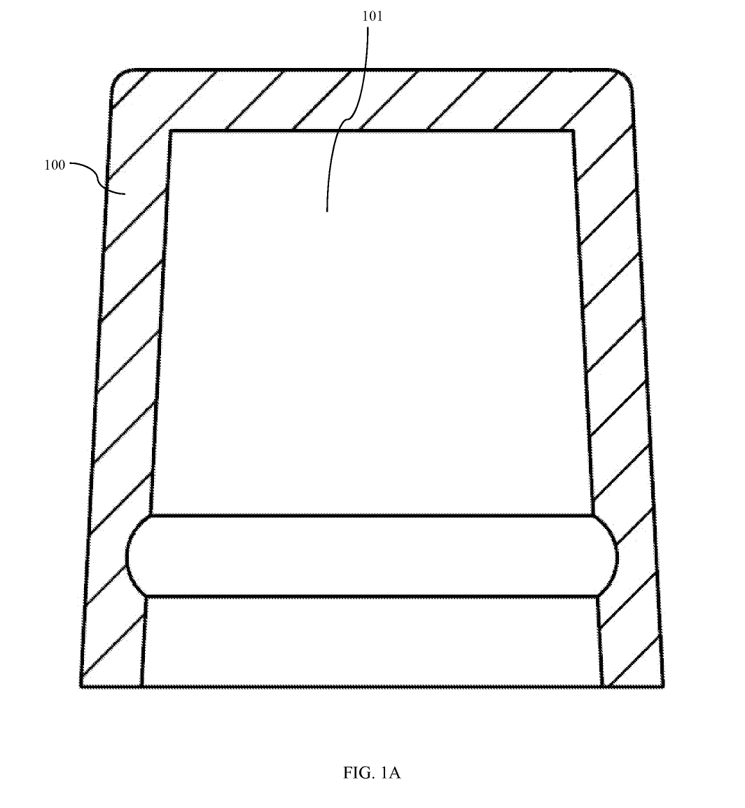

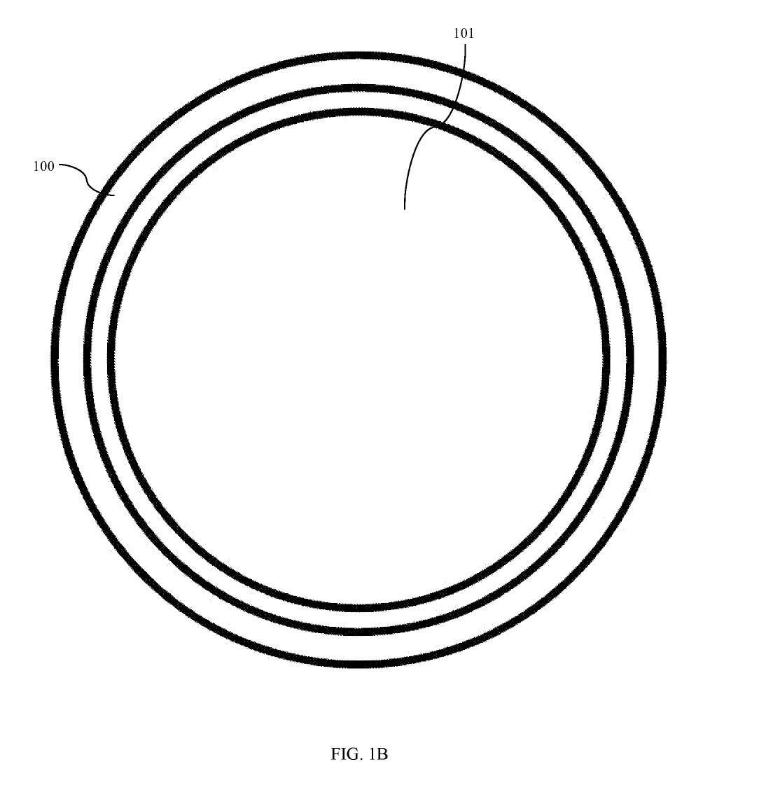

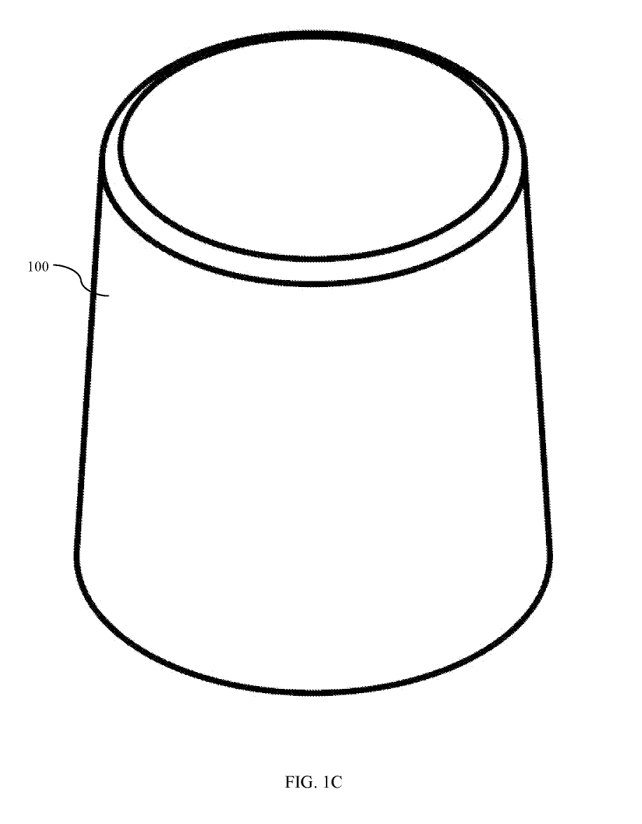

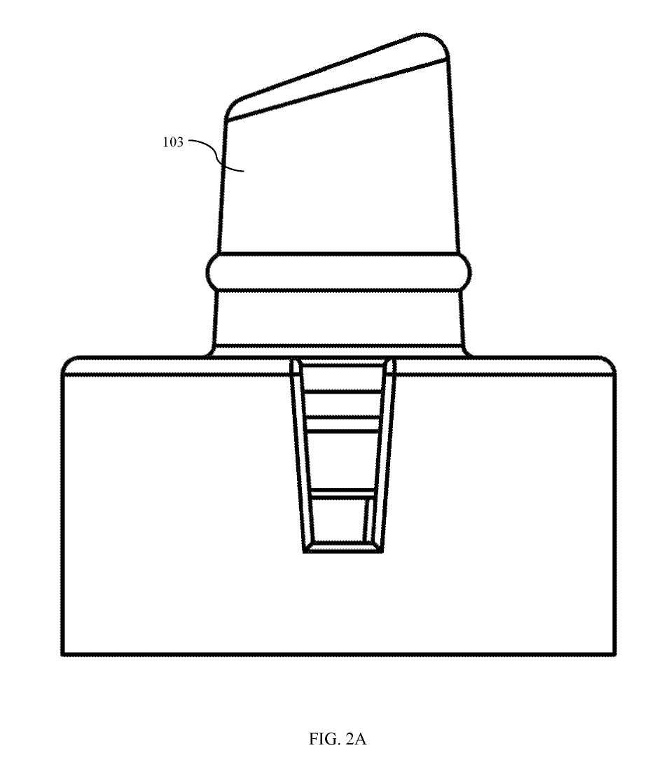

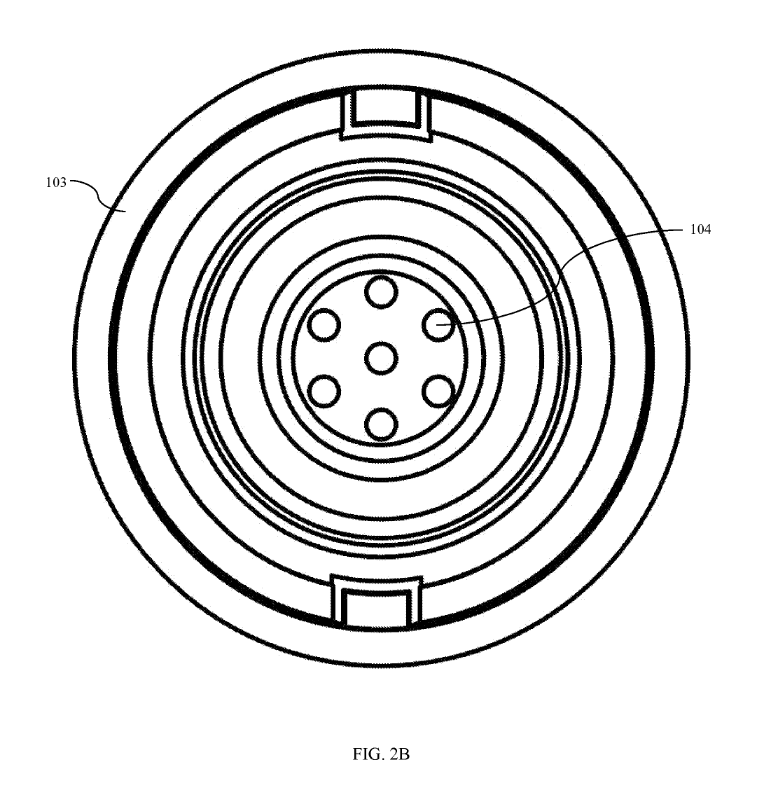

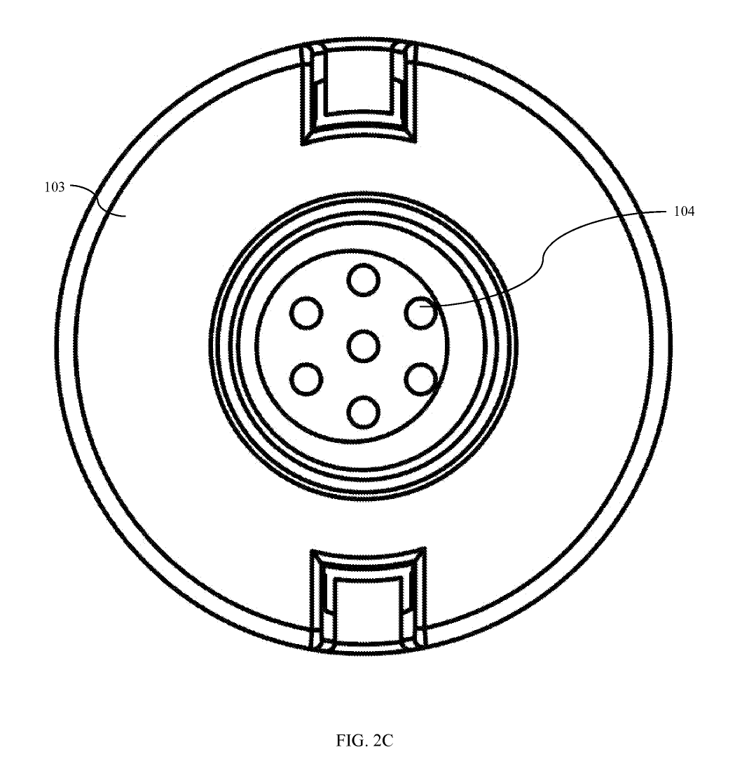

Referring now to FIGS. 1A-C that will be discussed together, there are shown side, bottom, and side elevated views of a screw cap (100) as according to one embodiment of the present invention. The screw cap (100) screws on to the applicator tip (FIGS. 2A-C, 9 (103)) to protect the applicator tip (FIGS. 2A-C, 9 (103)) when the medication applicator is not in use. The screw cap (100) has a screw cap internal opening (101) into which the applicator tip (FIGS. 2A-C, 9 (103)) fits when the screw cap (100) is placed on the applicator. Threads are located on the underside of the screw cap (100) so that the screw cap (100) can screw onto the applicator tip (FIGS. 2A-C, 9 (103)). Screwing or snapping the screw cap (100) onto the applicator tip (FIGS. 2A-C, 9 (103)) allows the screw cap (100) to securely attach to the applicator tip (FIGS. 2A-C, 9 (103)) and reduces the probability of accidental dislodgement.

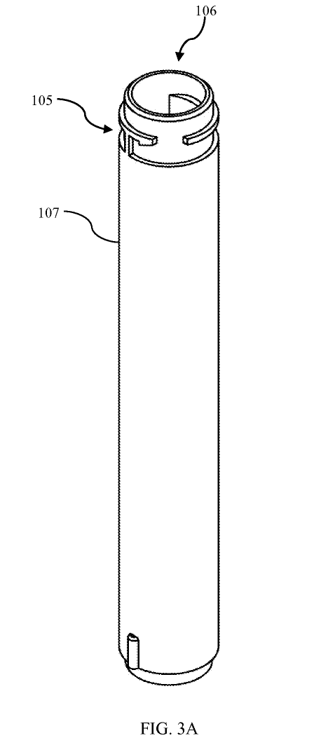

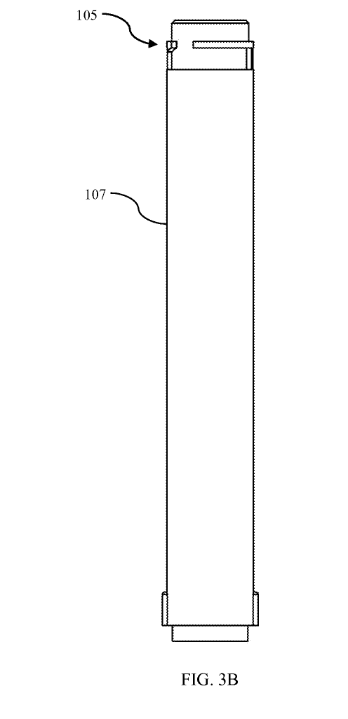

Referring now to FIGS. 2A-C that will be discussed together, there are shown side, bottom, and side elevated views of an applicator tip (103) as according to one embodiment of the present invention. The applicator tip (103) is located on top of the medication applicator barrel (FIGS. 3A-B, 9 (107)) and is the component of the medication applicator through which medication is dispensed. Applicator tip pores (104) in the applicator tip (103) allow liquid, cream, ointment, or other types of non-solid medication to exit when a user dispenses medication. The applicator tip (103) has an angled surface to allow for easier and more convenient distribution of the medication over a surface such as the skin of a user. The applicator tip (103) has threads on its underside so that the applicator tip (103) can screw on to the medication applicator barrel (FIGS. 3A-B, 9 (107)).

Referring now to FIGS. 3A and 3B that will be discussed together, there are shown side elevated view and side views of a medication applicator barrel (107) as according to one embodiment of the present invention. The medication applicator barrel (107) is the component of the medication applicator where medication is stored until dispensed. The medication applicator barrel is constructed of an opaque material so that light cannot damage the medication housed within the barrel. It is well-known that some medications lose their efficacy or can be damaged by UV exposure, or exposure to other light wavelengths. The opacity of the medication applicator barrel prevents this damage. However, the medication barrel is of sufficient translucence to allow users to see how much medication remains within the applicator.

The medication applicator barrel (107) is cylindrical in shape with an opening (106) at the top of the barrel. For purposes of this description it should be noted that the “top” of the medication applicator barrel (107) is considered to be the end of the medication applicator barrel (107) that is open and attaches to the applicator tip (FIGS. 2A-C, 9 (103)). A threaded area (105) is located on the exterior surface of the top of the medication applicator barrel (107) so that the applicator tip (FIGS. 2A-C, 9 (103)) can screw onto the medication applicator barrel (107). The top part of the medication applicator barrel (107) is where medication is stored. The medication applicator barrel (107) can store a volume of non-solid medication such as creams, ointments, lotions, liquids, gels, oils, balms, unctions, unguents, or liniments. These medications can be dispensed topically onto the skin of a patient, or orally as required. Any medication that does not require an injection-type of administration may be stored in the medication applicator barrel (107) and dispensed when needed.

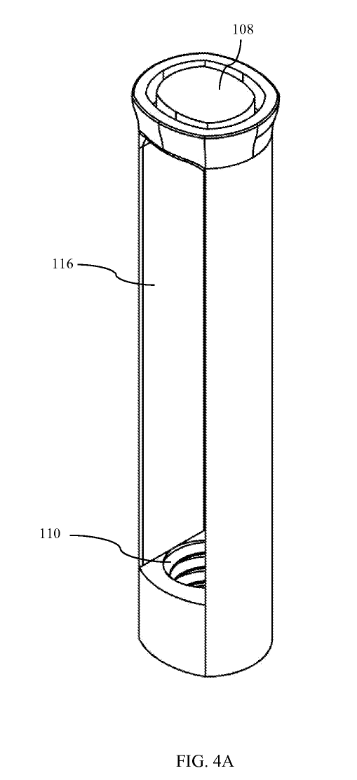

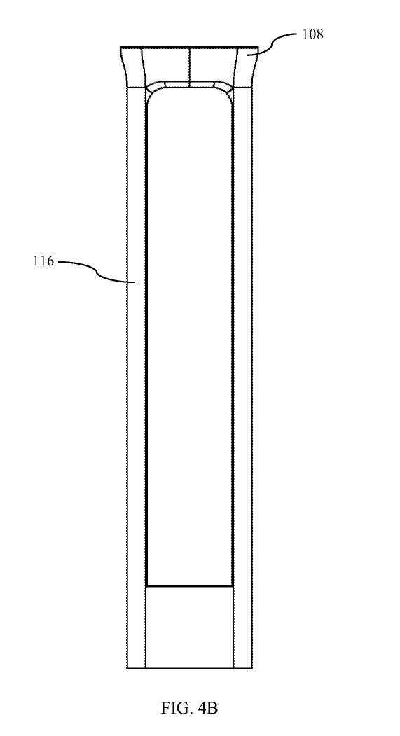

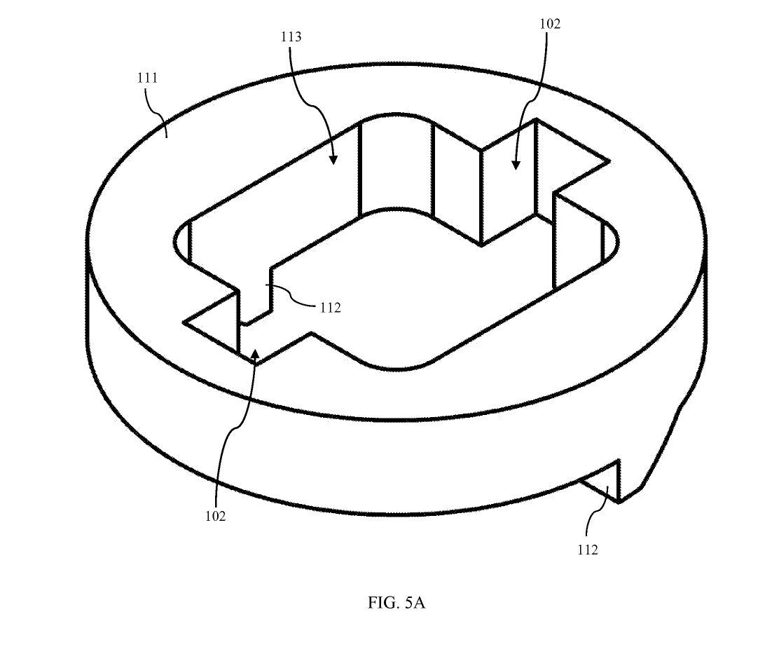

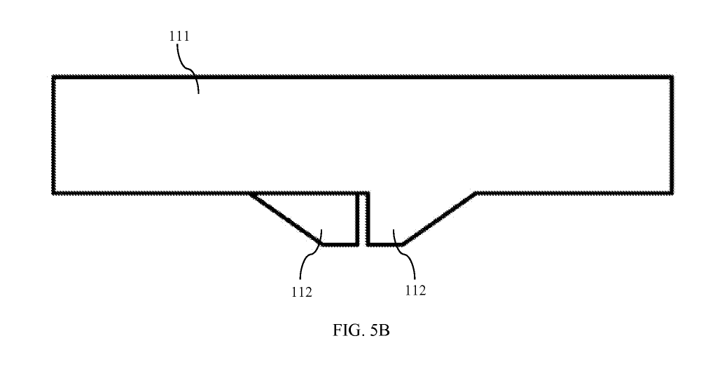

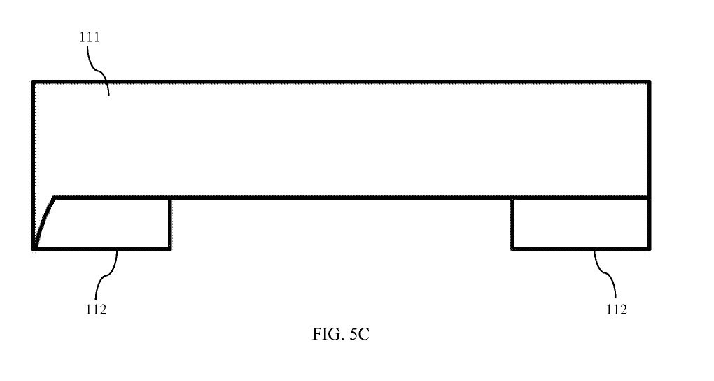

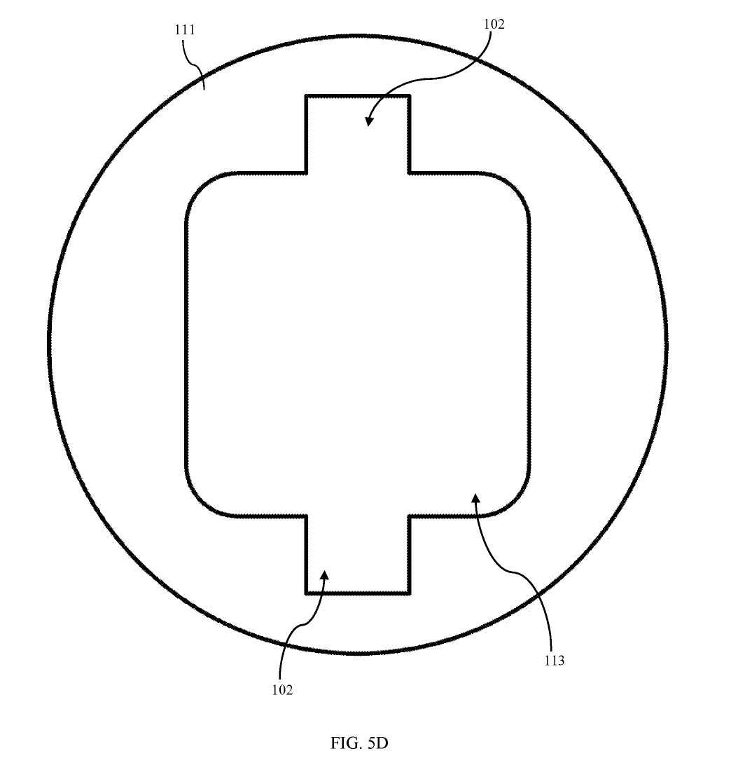

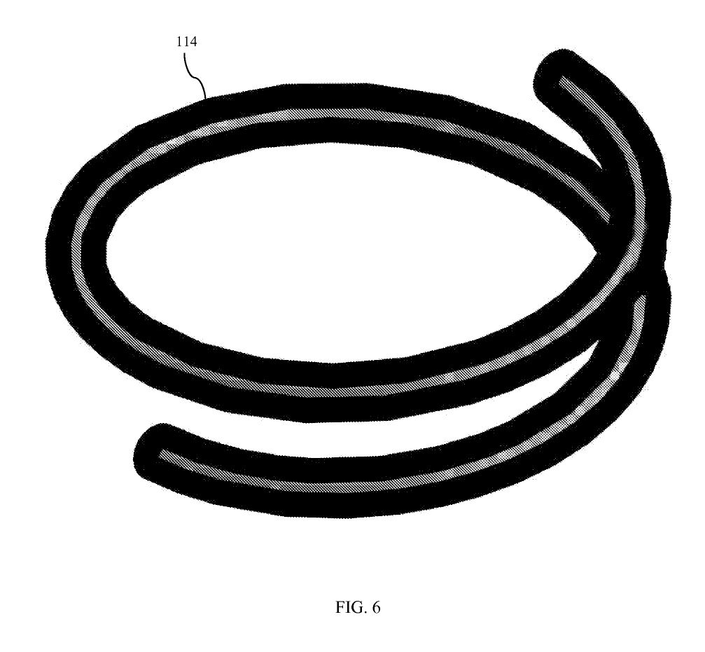

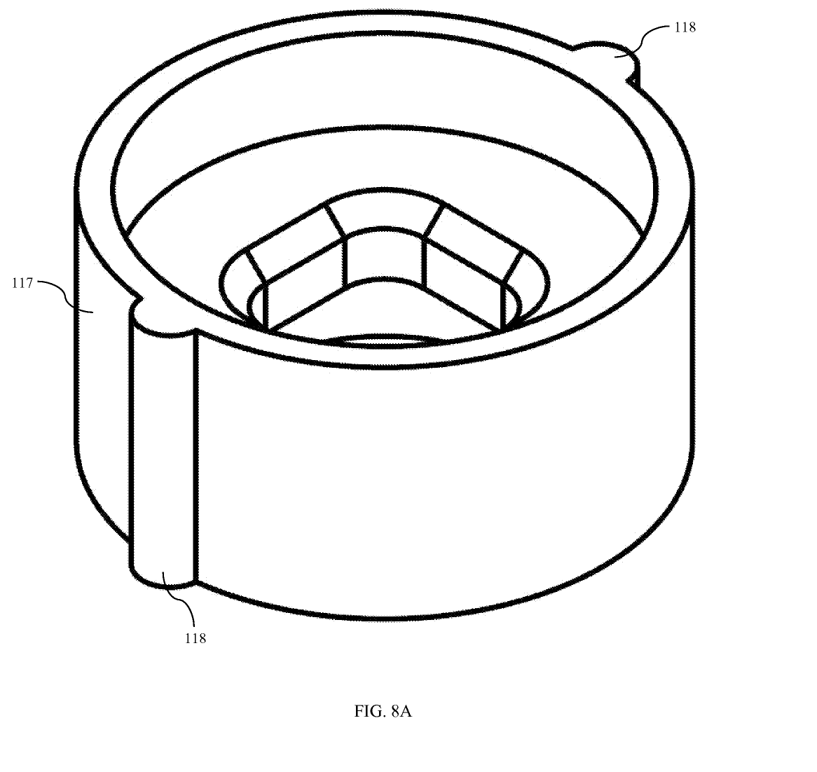

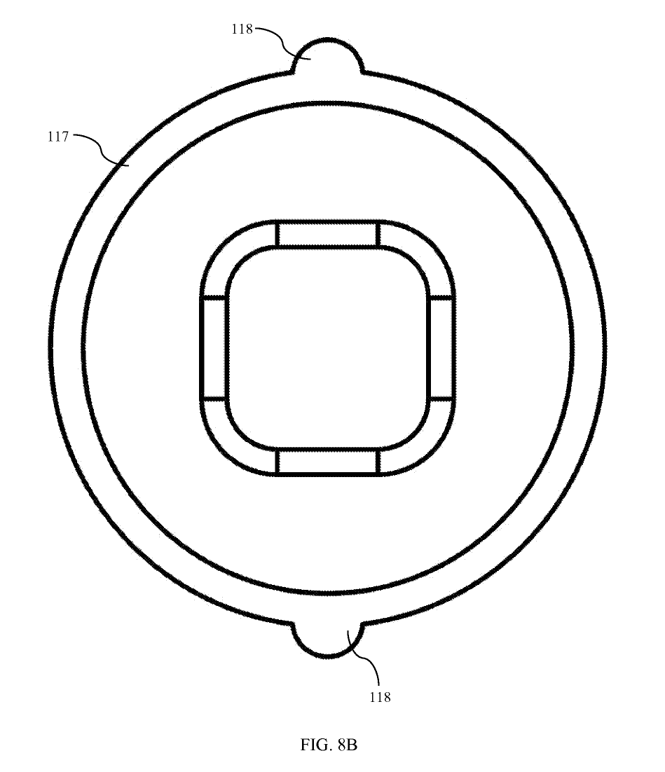

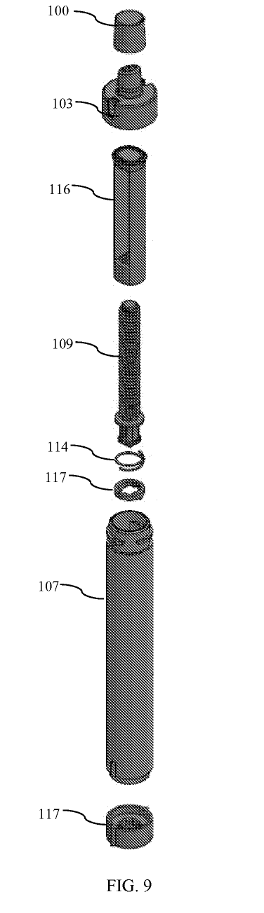

The medication applicator barrel (107) houses a plunger (FIGS. 4A-B, 9 (116)) that causes medication to exit the medication applicator during dispensation. For purposes of this description only it should be noted that the “bottom” of the medication applicator barrel (107) is considered to be the area of the medication applicator barrel (107) that is at the opposite end of the applicator barrel (107) from the applicator tip opening (106). The bottom of the medication applicator barrel (107) also houses the ratchet disk (FIGS. 5A-B, 9 (111)), spring (FIGS. 6, 9 (114)), and twist grip (FIGS. 8A-B, 9 (117)). The function and purpose of these aforementioned components is described in full below.

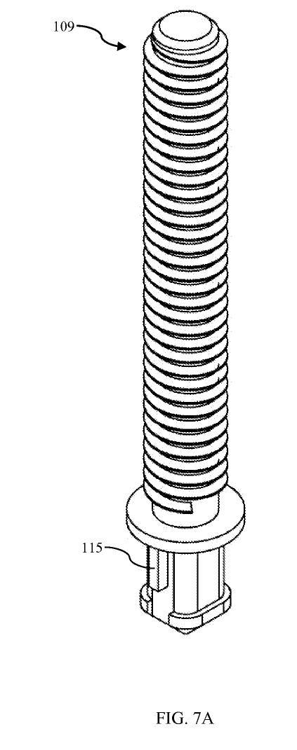

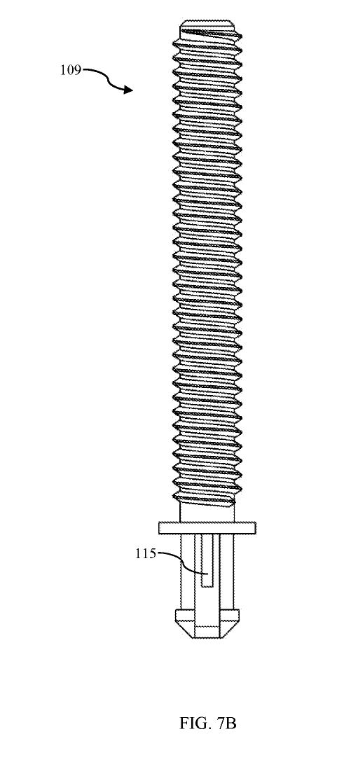

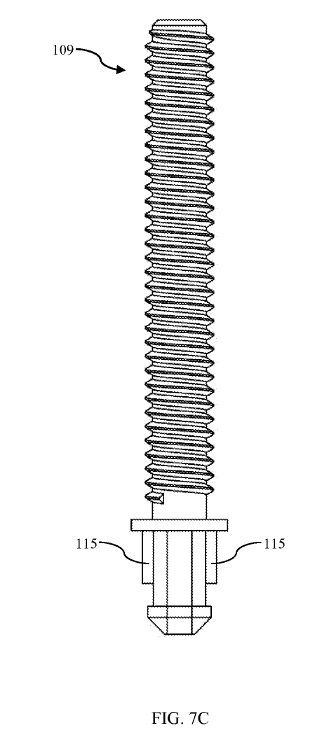

Referring now to FIGS. 4A and 4B, there is shown a side view of a plunger (116) as according to one embodiment of the present invention. The plunger (116) is located within the medication applicator barrel (FIGS. 3A-B, 9 (107)) and comprises a platform (108), and interior threaded area (110). The platform (108) contacts the medication stored in the medication applicator barrel (FIGS. 3A-B, 9 (107)). When the user dispenses medication, the twist grip (FIGS. 8A-B, 9 (117)) is pressed inwards, the ratchet disk teeth (FIGS. 5A-C (112)) engage with the twist grip (FIGS. 8A-B, 9 (117)). The user then rotates the twist grip (FIGS. 8A-B, 9 (117)). The rotation of the twist grip (FIGS. 8A-B, 9 (117)) causes a threaded rod (FIGS. 7A-C, 9 (109)) to rotate which, in turn, advances the plunger (116) in the medication applicator barrel (FIGS. 3A-B, 9 (107)). The platform (108) of the plunger forces medication out through the pores (FIGS. 2B-C (104)) of the applicator tip (FIGS. 2A-C, 9 (103)). The threaded rod (FIGS. 7A-C, 9 (109)) contacts the interior threaded area (110) and causes the plunger (116) to advance as the threaded rod (FIGS. 7A-C, 9 (109)) rotates. Advancement of the plunger (116) causes medication to be dispensed through the applicator tip (FIGS. 2A-C, 9 (103)).

In some embodiments of the present invention, the plunger (116) causes precisely 0.05 mL +/−5% of medication to be dispensed for every ½ rotation of the twist grip (FIGS. 8A-B, 9 (117)). The amount dispensed per rotation, or the rotational distance required to dispense medication may differ in other embodiments. By way of example, some embodiments of the present invention may dispense a precise dose of 0.10 mL +/−5% per ½ rotation of the twist grip (FIGS. 8A-B, 9 (117)) while other embodiments may dispense 0.05 mL +/−5% for every ½ rotation of the twist grip (FIGS. 8A-B, 9 (117)).

Referring now to FIGS. 5A-D, FIG. 6 and FIGS. 7A-C that will be described together, there are shown views of a ratchet disk (111), a spring (114), and a threaded rod (109). The ratchet disk (111) and spring (114) are housed in the medication applicator barrel (FIGS. 3A-B, 9 (107)). The plunger (FIGS. 4A-B, 9 (116)) passes through the interior opening of the ratchet disk (113) and spring (114). Alignment slots (102) are located within the ratchet disk (111). The alignment slots (102) receive the threaded rod alignment tabs (115). The mating of the alignment slots (102) and threaded rod alignment tabs (115) allow the threaded rod (109) to rotate when the ratchet disk (111) rotates.

The spring (114) resides between the ratchet disk (111) and the threaded rod (109). The spring (114) creates a clicking sound when the twist grip is rotated. The clicking sound audibly alerts users that medication is being dispensed. Furthermore, the clicking sound generated by the spring (114) lets users know they have rotated the twist grip the proper amount to dispense the correct dose of medication.

When the ratchet disk teeth (112) are engaged with the twist grip (FIGS. 8A-B, 9 (117)), rotation of the twist grip (FIGS. 8A-B, 9 (117)) causes the threaded rod (109) to rotate which further causes the plunger (FIGS. 4A-B, 9 (116)) to advance within the medication applicator barrel (FIGS. 3A-B, 9 (107)).

Referring now to FIGS. 8A and 8B, there is shown views of a twist grip (117) as according to one embodiment of the present invention. The twist grip (117) has finger threads (118) around the bottom portion so that the twist grip (117) does not slip between a user's fingers when the user is dispensing medication. To dispense medication, the twist grip (117) is pressed inwards so that the ratchet disk teeth (FIGS. 5A-C (112)) and twist grip (117) engage. Then, the twist grip (117) is rotated. The user precisely controls the amount of medication being dispensed by determining how far to rotate the twist grip (117). By rotating the twist grip (117) in exact amounts, a precise measurement of medication is dispensed. In some embodiments of the present invention, a half turn is required for a minimum dose. The user may rotate the grip (117) more than a half turn if more medication is desired. The twist grip (117) houses the end of the threaded rod (FIGS. 7A-C, 9 (109)).

Referring now to FIG. 9, there is shown a component view of the assembly of a medication applicator as according to one embodiment of the present invention. A screw cap (100) fits onto an applicator tip (103). A plunger (116), threaded rod (109), spring (114), and ratchet disk (117) reside within an applicator barrel (107). Medication is also housed within the applicator barrel (107) and is extruded from the applicator tip (103) as the threaded rod (109) rotates and pushes the plunger (116) toward the applicator tip (103). A twist grip (117) is used by a user to set the dosage amount.

Referring now to operation of the medication applicator, during an initial stage of dispensation, medication fills a medication reservoir in the applicator barrel. The plunger is fully compressed within the twist grip and the plunger platform is at the bottom of the reservoir. As medication is dispensed, the twist grip is rotated and the threaded rod of the plunger travels upward through the ratchet disk interior opening and spring. The plunger traverses upward within the medication reservoir toward the applicator tip causing medication to be dispensed from the tip. When all of the medication has been dispensed, the platform is at the top of the medication reservoir.

Some embodiments of the present are single-use and are considered to be “disposable”. Once the initial amount of medication is exhausted, the medication applicator is discarded.

The medication applicator is easy and relatively low-cost to manufacture. The low-cost nature of the medication applicator makes it an ideal solution for economically dispensing topical or oral medication and greatly enhances its commercial viability.

Embodiments of the present invention are constructed from a material that possesses important properties. Embodiments are constructed from materials that may possess any combination of, or none of, the following properties: highly durable, rugged, lightweight, UV-resistant, non-conductive, and temperature resistant. Some materials that possess one or more of these properties that may be used to construct the medication applicator include, but are not limited to, polymers, plastics, metals, or alloys. The medication applicator may be constructed from any single or combination of the aforementioned materials.

The above detailed description sets forth rather broadly the more important features of the present invention in order that its contributions to the art may be better appreciated.

As set forth in this description and the attached images, an improved medication applicator has been developed that improves upon conventional syringe-type medication applicators. The various embodiments of the improved medication applicators described herein can be used to dispense a wide variety of medications.

The preceding exemplary embodiments are not intended to be limiting, but are merely illustrative for the possible uses of the medication applicator.

As such, those skilled in the art will appreciate that the conception, upon which disclosure is based, may readily be utilized as a basis for designing other structures, methods, and systems for carrying out the several purposes of the present invention. It is important, therefore, that this description be regarded as including such equivalent constructions insofar as they do not depart from the spirit and scope of the present invention.

Although certain example methods, apparatus and articles of manufacture have been described herein, the scope of coverage of this provisional patent application is not limited thereto. On the contrary, this provisional patent application covers all methods, apparatus and articles of manufacture fairly falling within the scope of the invention either literally or under the doctrine of equivalents.

At least some of the above described example methods and/or apparatus may be designed, tested, or manufactured by one or more software and/or firmware programs running on a computer processor. However, dedicated hardware implementations including, but not limited to, an ASIC, programmable logic arrays and other hardware devices can likewise be constructed to implement some or all of the example methods and/or apparatus described herein, either in whole or in part.

To the extent the above specification describes example components and functions with reference to particular compliance requirements, standards and/or protocols, it is understood that the teachings of this disclosure are not limited to such compliance requirements, standards and/or protocols. Such compliance requirements, standards and/or protocols are periodically superseded or revised by newer versions. Accordingly, replacement compliance requirements, standards and/or protocols having the same general functions are equivalents which are intended to be included within the scope of this description.

Directional terms such as “front”, “forward”, “back”, “rear”, “in”, “out”, “downward”, “upper”, “lower”, “top”, “bottom”, and the like may have been used in the description. These terms are applicable to the embodiments shown and described herein. These terms are merely used for the purpose of description and do not necessarily apply to the position in which components or items within the present invention may be used.

Therefore, the foregoing is considered as illustrative only of the principles of a medication applicator. Further, since numerous modifications and changes will readily occur to those skilled in the art, it is not desired to limit the medication applicator to the exact construction and operation described, and accordingly, all suitable modifications and equivalents may be resorted to, falling within the scope present invention. While the above description describes various embodiments of the present invention, it will be clear that the present invention may be otherwise easily adapted to fit any configuration where using a medication applicator is desired or required.

As various changes could be made in the above constructions without departing from the scope of the invention, it is intended that all matter contained in the above description shall be interpreted as illustrative and not in a limiting sense.

I claim:

1. An apparatus for dispensing oral or topical medication comprising:
an applicator tip, pores in the applicator tip through which medication passes when the medication is dispensed, a medication applicator barrel that houses the medication, a plunger that consists of a platform and an interior threaded area, the platform causing the medication to exit the apparatus through the pores in the applicator tip when the medication is dispensed, a threaded rod that causes the plunger to advance when the threaded rod is rotated, a ratchet disk with teeth that contacts a twist grip, where rotation of the twist grip causes the threaded rod to rotate within the medication applicator barrel when the medication is dispensed, and where rotation of the twist grip causes precise doses of medication to be dispensed.

2. The apparatus of claim 1, wherein the apparatus further comprises a cap that attaches to the applicator tip in order to cover and protect the applicator tip when medication is not being dispensed.

3. The apparatus of claim 1, wherein the applicator tip has an angled surface to allow for easier and more convenient distribution of medication.

4. The apparatus of claim 1, wherein the twist grip is rotated by a user in order to cause the ratchet disk and twist grip engage.

5. The apparatus of claim 1, wherein less than one full rotation of the twist grip is required to dispense a minimum dose of medication.

6. The apparatus of claim 1, wherein the medication applicator barrel allows users to visually observe the amount of remaining medication.

7. The apparatus of claim 1, wherein the apparatus is disposable and is discarded after the store of medication has been exhausted.

8. A medication dispensation apparatus that provides precise doses of topical or oral medication comprising:
a medication applicator barrel that stores topical or oral medication, a twist grip that controls the amount of the medication to be dispensed by providing a precise dose of medication when the twist grip is rotated; a ratchet disk that causes a threaded rod within the apparatus to rotate when the twist grip is rotated; the a plunger comprising a platform that directly contacts the medication and causes the medication to exit from pores in an applicator tip during dispensation, the applicator tip being located at one end of the medication applicator barrel; wherein the threaded rod contacts the plunger and contacts the twist grip; wherein rotation of the twist grip causes the threaded rod to rotate which, in turn, causes the plunger to advance within the apparatus; the ratchet disk and twist grip each having an opening through which a portion of the threaded rod is housed.

9. The medication dispensation apparatus of claim 8, wherein finger threads are located on the twist grip that prevent slippage of a user's fingers when the twist grip is turned.

10. The medication dispensation apparatus of claim 9, wherein the finger threads provide a visual indicator of alignment of the twist grip and medication applicator barrel.

11. The medication dispensation apparatus of claim 8, wherein the medication applicator barrel is opaque and prevents light from damaging the medication within the apparatus.

12. The medication dispensation apparatus of claim 8, further comprising a screw cap that screws on to the applicator tip to protect the applicator tip.

13. The medication dispensation apparatus of claim 8, wherein a minimum dispensed dose is 0.05 mL plus or minus 5%.

14. A device for dispensing exact doses of topical or oral medication comprising:
a means for housing topical or oral medication, a user-controlled means for setting a precise amount of medication to be dispensed, a means for dispensing the precise amount of medication, a means for protecting the medication from light damage; where the means for housing topical or oral medication is a medication reservoir area within a medication applicator barrel; the user-controlled means for setting a precise amount of medication to be dispensed is a rotatable twist grip that is connected to a threaded rod; the means for dispensing the precise amount of medication is the rotation of the twist grip, the rotation of the twist grip causing the precise amount medication to be dispensed;
wherein twisting the twist grip causes the twist grip to engage with a ratchet disk that is within the medication applicator barrel, the ratchet disk having alignment slots that are in contact with alignment tabs on a threaded rod, and the threaded rod being attached to a plunger that forces medication out through an applicator tip when medication is dispensed, the plunger travelling within the medication applicator barrel toward an applicator tip when the twist grip is rotated.

15. The device of claim 14, wherein the medication applicator barrel further comprises a threaded area for attaching the medication applicator barrel to the applicator tip.

16. The device of claim 14, wherein the means for means for protecting the medication from light damage comprises the medication barrel being constructed from an opaque material.

17. The device of claim 14, further comprising a spring that creates a clicking sound when the twist grip has been rotated sufficiently.

18. The device of claim 14, wherein a half-turn of the twist grip is required to dispense a minimum dose.

19. The device of claim 14, wherein dose amounts can be administered in increments of 0.05 mL plus or minus 5%.

20. The device of claim 14, wherein the device is single-use and is discarded once an initial store of medication is exhausted.
